(19) United States
(12) Patent Application Publication
(10) Pub. No.: US 2007/0265657 A1
(43) Pub. Date: Nov. 15, 2007

(54) Box joint medical tool apparatus and method of manufacture

(76) Inventor: Anthony J. Sexton, Racine, WI (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 11/431,481

(22) Filed: May 11, 2006

(51) Int. Cl. A61B 17/00

(52) U.S. Cl. 606/208; 606/205

(57) ABSTRACT

An embodiment in accordance with the present invention provides a medical forceps having an improved method of manufacture thereof. The method of the present invention includes a method of forming, assembling and manufacturing a medical tool, such as a medical forceps, which has a box joint, the method eliminating the need for initial assembly of the box joint through spreading apart the sidewalls of the female box joint portion. The present invention thereby allows for better cleaning and polishing of the male and female pivot areas around the box joint portion prior to final assembly of the device.

### CROSS-REFERENCE TO RELATED APPLICATIONS

None.

### FIELD OF THE INVENTION

The present invention relates generally to medical devices. More particularly, the present invention relates to a method of manufacturing a medical instrument having a box joint, and an apparatus corresponding thereto.

### BACKGROUND OF THE INVENTION

Box joints are a commonly employed coupling and hinge mechanism used in a variety of tools. Included among these tools are various medical instruments, such as a forceps, scissors, or a rongeur. An example of a box joint used in a medical instrument such as a forceps is shown in FIG. 1, which depicts a box joint 1 which couples the handle portions 2,3 of two body parts 4,5 of a forceps (the distal operating end portions opposite the handle portions not being shown). In a box joint configuration, one body part or half of the device, commonly referred to as the “female” portion, includes a channel 10 that is part of the box joint portion mechanism, as shown in FIG. 2. The channel 10 is sized to be just wide enough to accommodate the neck of a complementary male body part or other half of the device, where such neck is arranged to be disposed through the channel 10 so that the two body parts cooperate in a hinge-like manner through the box joint mechanism, as is well known to those skilled in the art.

The general approach to manufacturing such a box jointed forceps is to first forge the left and right blanks which will form the two body parts of the device. The blanks are then machined to size and shape, as dictated by the particular tool to be manufactured. However this method of manufacture and assembly can be particularly difficult for and problematic for box joint type instruments. According to the known prior art, and with reference to the example shown in FIG. 2, the only way to assemble and manufacture a box joint type instrument is to heat the female portion 5 with a suitable heat source, such as, for example, a propane torch, and to then spread the walls 12 and 14 forming the pivot area around channel 10 in the direction of arrows A, and to then insert the male portion 2 through the opening 10 and then hammer the walls 12 and 14 of the female portion 3 back into shape. However this part of the assembly process has to done early in the manufacturing cycle, which means that when the finished instrument needs to be cleaned and polished it is impossible to clean and polish down in the box joint pivot area 1.

Accordingly, it is desirable to provide an improved method of forming, assembling, and manufacturing a medical instrument having a box joint. It is further desirable that the improved method of making a box joint in a medical instrument reduces the costs of manufacture and improves the overall efficiency of the manufacturing process for the medical instrument. It is further desirable to provide an improved method of manufacturing, assembling and finishing a medical device having a box joint where said method will eliminate the need for early assembly of the box joint as elaborated above and will instead allow for better cleaning and polishing of the male and female pivot areas around the box joint portion prior to final assembly of the device.

### SUMMARY OF THE INVENTION

The foregoing needs are met, to a great extent, by the present invention, wherein in one aspect an apparatus is provided that in some embodiments provides an improved method of manufacturing, assembling and finishing a medical device having a box joint where said method will eliminate the need for early assembly of the box joint and will instead allow for better cleaning and polishing of the male and female pivot areas around the box joint portion prior to final assembly of the device.

In accordance with one embodiment of the present invention, a method of assembling a medical instrument having a box joint is provided. A first half-body is formed having a female box joint portion between a first handle portion and a first operating end portion. The female box joint portion defines a channel through the first half-body. A second handle portion is formed. And a second operating end portion is formed. The second operating end portion includes a male box joint portion complementary to the female box joint portion. The male box joint portion includes a first mating end configured to mate with a complementary second mating end defined by the second handle portion. The male box joint portion is inserted through the channel in the female box joint portion. The first and second mating ends are coupled to join the second handle portion and second operating end portion to form the second half-body.

In accordance with another aspect of the present invention, a medical instrument is provided, which includes a first half-body and a second half-body joined by a box joint. The first half-body includes a female box joint portion defining a channel for accommodating a male box joint portion in the second half body. The second half-body is formed by at least two pieces including a first piece and a second piece. Each said piece includes a complementary mating means for being coupled to the other piece. The first piece incorporates the male box joint portion of the device. The instrument is formed by inserting the male box joint portion and mating means of the first piece of the second half-body through the channel in the first half-body and then joining the first and second pieces of the second half-body by permanently coupling the complementary mating means on the first and second pieces.

In accordance with yet another aspect of the present invention, a medical instrument having a box joint is provided, and includes a first half-body having a female box joint portion between a first handle portion and a first operating end portion. The female box joint portion defines a channel through the first half-body. A second half-body is formed by a second handle portion and a second operating end portion. The second operating end portion includes a male box joint portion complementary to the female box joint portion. The male box joint portion includes a first mating element configured to mate with a complementary second mating element defined by the second handle portion. The male box joint portion is inserted through the channel in the female box joint portion prior to coupling the first and second mating elements to join the second handle portion and second operating end portion to form the second half-body and thereby assemble the medical instrument.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a plan view showing a medical instrument with a box joint.

FIG. 2 is a bottom view showing the female portion of the medical instrument illustrated in FIG. 1.

FIG. 3 is a front view of the medical instrument shown in FIG. 1.

FIG. 3A is an enlarged front view of the area A around the box joint of the medical instrument shown in FIG. 3.

FIG. 4 is a perspective view of a female portion of a medical instrument in one embodiment of the present invention.

FIG. 5 is a perspective view of two portions which form the male portion of the medical instrument according to the present invention.

### DETAILED DESCRIPTION

The invention will now be described with reference to the drawing figures, in which like reference numerals refer to like parts throughout. An embodiment in accordance with the present invention provides a medical forceps having an improved method of manufacture thereof. The method of the present invention includes a method of forming, assembling and manufacturing a medical tool, such as a medical forceps, which has a box joint, the method eliminating the need for initial assembly of the box joint through spreading apart the sidewalls of the female box joint portion. The present invention thereby allows for better cleaning and polishing of the male and female pivot areas around the box joint portion prior to final assembly of the device.

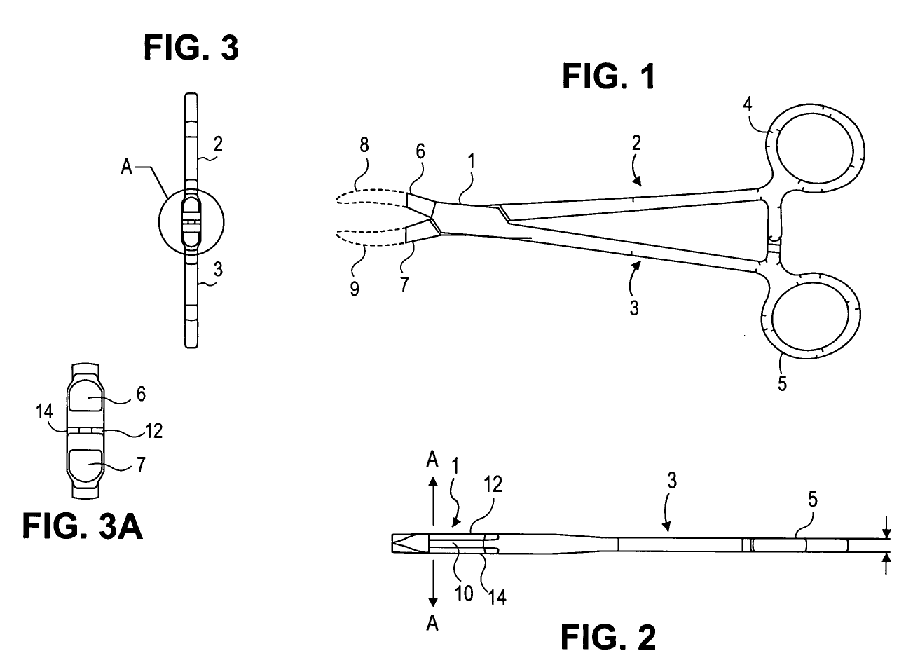

Turning now to the figures, FIG. 1 shows a plan view of a medical instrument, such as a forceps, having a box joint. Such a box joint is well-known to those of ordinary skill in the art. The box joint 1 shown in FIG. 1 joins two pieces of the tool, which are referred to herein as a “half-body.” The tool can include a first “male” half-body 2 and a second “female” half-body 3, which are conjoined at the box joint portion 1. Each of the male and female half-bodies 2 and 3 can have handle portions 4 and 5 at their proximal ends. In accordance with conventional practice, as used herein, the term “proximal” or “proximal end” shall refer to the specified end of a device or its component which is generally closer to the medical personnel handling or manipulating the device as it is intended to be used, and the term “distal” or “distal end” shall refer to the specified end of a device or its component which is opposite the proximal end. The medical instrument therefore includes distal first and second operating end portions 6 and 7. Each of the first and second operating end portions 6 and 7 can have any of a variety of well-known distal extensions 8 and 9, respectively, which are shown in FIG. 1 as dotted lines so that the scope of the present invention is understood to include any medical instrument having any distal operating end, such as, for example, a forceps.

FIG. 2 is a top view showing the female portion of the medical instrument illustrated in FIG. 1. The box joint portion 1 is shown with the male half-body's neck portion 10 which fits between the two sidewalls 12 and 14 that make up the female portion of the box joint defined by the female half-body 3. The two sidewalls 12 and 14 therefore define a channel into which the male neck portion 10 fits to produce a snug box joint interconnection. Conventional assembly of such a box joint involves the initial spreading apart of the two sidewalls 12 and 14 in the direction of arrows “A” shown in FIG. 2, after which the male half-body 2 is then inserted between the two sidewalls 12 and 14 which are then reformed and straightened to close the box joint. FIG. 3 is a front view of the medical instrument shown in FIG. 1. FIG. 3A is an enlarged front view of the area A around the box joint of the medical instrument shown in FIG. 3.

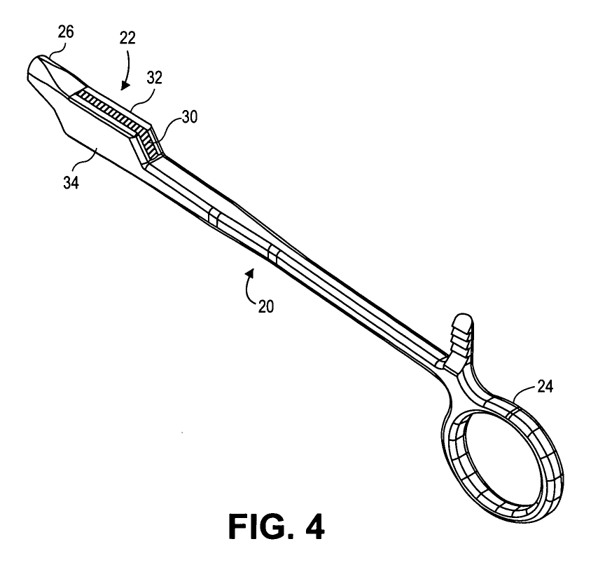

The present invention provides a method of assembly and resulting medical tool having a box joint, whereby the conventional step of spreading apart the box joint's female wall portions is obviated by the inventive principles disclosed herein. FIG. 4 is a perspective view of a female portion or half-body 20 of a medical instrument in one embodiment of the present invention. The female half-body 20 includes a female box joint portion 22 interposed between a proximal handle portion 24 and a distal operating end portion 26 (extreme distal tip not shown). A channel 30 is shown between the two sidewalls 32 and 34 of the female box joint portion 22.

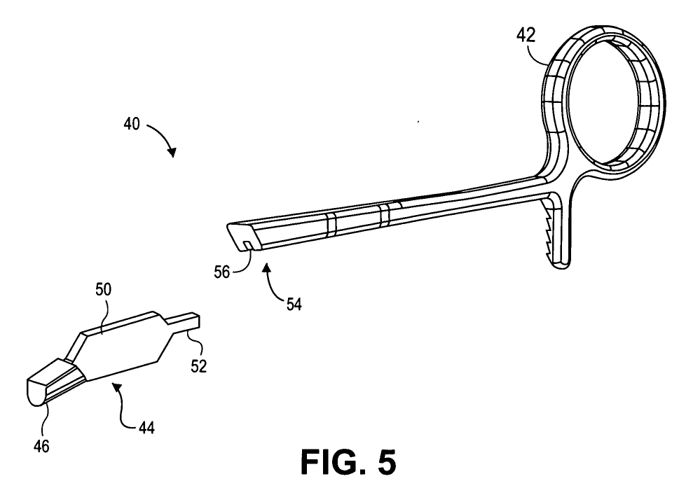

FIG. 5 is a perspective view of two portions which form the male portion or second half-body 40 of the medical instrument according to the present invention. The second half-body 40 includes two pieces, a second proximal handle portion 42 of the medical instrument, and a male box joint portion 44 having a distal operating end portion 46 (extreme distal tip not shown). The male box joint portion 44 includes a neck portion 50 which can generally be a narrow, thin planar element configured to snugly fit within the channel 30 defined by the female box joint portion 22. As such, the male box joint portion 44 is complementary to the female box joint portion 22 so as to form a properly interconnected and operating box joint for the medical instrument.

The male box joint portion 44 also includes a first mating end portion 52. Mating end 52 is shown in the embodiment in FIG. 5 as a finger-like extension or protrusion on the proximal end of the male box joint portion 44, extending from the planar neck portion 50. Mating end 52 is configured and shaped to fit into a corresponding and complementary second mating end 54 defined at the distal end of the handle portion 42 of the male half-body 40. Complementary second mating end 54 is defined in the embodiment shown in FIG. 5 as a narrow slot 56 into which the finger-like protrusion 52 can fit. Interconnection of the two complementary mating ends 52 and 54 can thereby couple the two pieces 42 and 44 of the male half-body.

According to the present invention, a medical device having a box joint is assembled by first making or machining the male portion 40 of the instrument in two pieces 42 and 44, such as shown in FIG. 5, as well as the female half-body 20 as shown in FIG. 4. The surface areas of both the male and female pieces and elements can be cleaned and polished properly and easily before assembly, especially around the critical box joint portions. The instrument can then be assembled by sliding the male box joint portion 44 through the channel 30 of the female box joint portion 22. Then the slot 56 in the male handle portion 42 can be aligned to the corresponding protrusion 52 on the male box joint portion 44, and both parts 42 and 44 can be permanently coupled together. Such coupling can be performed in a myriad of ways, but, in a preferred embodiment, can be accomplished by resistance welding. Resistance welding can be much stronger than laser welding or some other form of welding, because the three surfaces on the protrusion 52 which are in apposition with the corresponding surfaces of slot 56 will be completely welded together providing a stronger bonded joint. The method of the present invention therefore would allow for very intricate distal tips on the instrument to be precision investment cast, regardless of the volumes, at a very cost effective rate. It is understood that the complementary mating means made up of elements 52 and 54,56 of the embodiment shown in FIG. 5 are but one form of mating and coupling the two pieces 42 and 44 of the male half-body 40, and that any number of alternative mating means can be incorporated into the present invention.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A method of assembling a medical instrument having a box joint, comprising:
forming a first half-body having a female box joint portion between a first handle portion and a first operating end portion, the female box joint portion defining a channel through the first half-body;
forming a second handle portion;
forming a second operating end portion, the second operating end portion including a male box joint portion complementary to the female box joint portion, the male box joint portion having a first mating end configured to mate with a complementary second mating end defined by the second handle portion;
inserting the male box joint portion through the channel in the female box joint portion,
coupling the first and second mating ends to join the second handle portion and second operating end portion to form the second half-body.

2. The method of claim 1,
wherein the medical instrument is a forceps.

3. The method of claim 1,
wherein the step of coupling the first and second mating ends to join the second handle portion and second operating end portion comprises resistance welding.

4. The method of claim 1,
wherein the first mating end comprises an elongate protrusion adapted to fit into a corresponding slot defined by the second mating end in the second handle portion.

5. The method of claim 4, wherein the step of coupling the first and second mating ends to join the second handle portion and second operating end portion comprises resistance welding.

6. A medical instrument, comprising:
a first half-body and a second half-body joined by a box joint,
the first half-body having a female box joint portion defining a channel for accommodating a male box joint portion in the second half body,
the second half-body being formed by at least two pieces including a first piece and a second piece, each piece having a complementary mating means for being coupled to the other piece, the first piece incorporating the male box joint portion,
wherein the instrument is formed by inserting the male box joint portion and mating means of the first piece of the second half-body through the channel in the first half-body and then joining the first and second pieces of the second half-body by permanently coupling the complementary mating means on the first and second pieces.

7. The medical instrument of claim 6,
wherein the medical instrument is a forceps.

8. The medical instrument of claim 6,
wherein the step of joining the first and second pieces of the second half-body by permanently coupling the complementary mating means on the first and second pieces comprises resistance welding.

9. The medical instrument of claim 6,
wherein the mating means on one of the at least two pieces includes an elongate protruding element, the elongate protruding element being shaped to fit into a corresponding slot defined by the mating means in another of the at least two pieces.

10. The medical instrument of claim 6,
wherein the step of joining the first and second pieces of the second half-body by permanently coupling the complementary mating means on the first and second pieces comprises resistance welding.

11. A medical instrument having a box joint, comprising:
a first half-body having a female box joint portion between a first handle portion and a first operating end portion, the female box joint portion defining a channel through the first half-body; and
a second half-body formed by a second handle portion and a second operating end portion, the second operating end portion including a male box joint portion complementary to the female box joint portion, the male box joint portion having a first mating element configured to mate with a complementary second mating element defined by the second handle portion,
the male box joint portion being inserted through the channel in the female box joint portion prior to coupling the first and second mating elements to join the second handle portion and second operating end portion to form the second half-body and assemble the medical instrument.

12. The medical instrument of claim 11,
wherein the medical instrument is a forceps.

13. The medical instrument of claim 11,
wherein the first and second mating elements are coupled by resistance welding to join the second handle portion and second operating end portion.

14. The medical instrument of claim 11,
wherein the first mating element includes an elongate protruding element, the elongate protruding element being shaped to fit into a corresponding slot defined by the mating means in another of the at least two pieces.

15. The medical instrument of claim 14,
wherein the first and second mating elements are coupled by resistance welding to join the second handle portion and second operating end portion.
